(19) United States
(12) Patent Application Publication
(10) Pub. No.: US 2008/0161561 A1
(43) Pub. Date: Jul. 3, 2008

(54) Novel Processing for the Preparation of a Benzofuran

(75) Inventors: Peter Schneider, Bottmingen (CH); Chouaib Tahtaoui, Rixheim (FR); Martin Braun, Shanghai (CN); Sorana Greiveldinger-Poenaru, Rheinfelden (CH); Jurgen Jaeger, Reinach (CH); Laurent Schmitt, Village Neuf (FR)

Correspondence Address: DICKSTEIN SHAPIRO LLP, 1177 AVENUE OF THE AMERICAS (6TH AVENUE), NEW YORK, NY, 10036-2714, US

(73) Assignee: Arpida, Reinach (CH)

(21) Appl. No.: 11/816,157

(22) PCT Filed: Feb. 10, 2006
(86) PCT No.: PCT/EP06/01179

§ 371 Date: Feb. 15, 2008

(30) Foreign Application Priority Data
Feb. 18, 2005 (EP) EP/2005/001695

(51) Int. Cl. C07D 239/24

(52) U.S. Cl. 544/297

(57) ABSTRACT

The present invention relates to a novel process for the preparation of the compound of formula I, a dihydrofolate reductase inhibitor

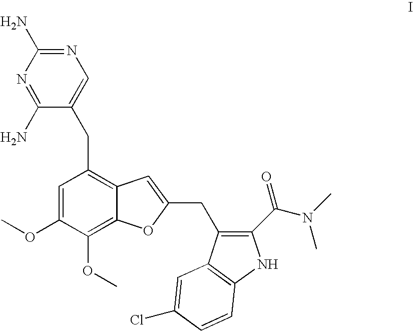

and to valuable intermediates in this process.

### FIELD OF THE INVENTION

The present invention relates to novel processes for the preparation of a compound of formula 1, which compound is related to dihydrofolate reductase inhibitors

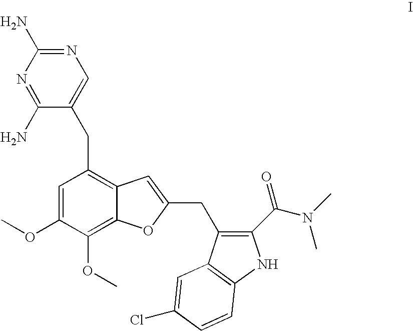

and to valuable intermediates in this process.

### BACKGROUND OF THE INVENTION

The compound of formula I has valuable antibiotic properties. The compound can be used in the control or prevention of infectious diseases in mammals, both humans and non-humans. In particular, it exhibits pronounced antibacterial activity, even against multiresistant Gram-positive strains and against opportunistic pathogens such as e.g. Pneumocystis carinii. The compound can also be administered in combination with known substances of antibacterial activity and exhibits synergistic effects with some of them.

Typical combination partners are e.g. sulfonamides or other inhibitors of enzymes, which are involved in folic acid biosynthesis such as, for example, pteridine derivatives.

Current method of preparing the compound of formula I is described in the patent application PCT/EP 2004/007482. The main drawback of this method is the lengthy synthesis and consequently the low overall yield. Most of the intermediates are not crystalline, which renders this synthesis economically less attractive for preparing commercial quantities. In addition, some expensive reagents cannot be recovered.

Therefore, there is a need for a process for preparing the compound of formula I with a higher overall yield and reduced number of intermediates, which can be isolated and purified. The aim is a process where all isolated intermediates are crystalline and do not require chromatography. In addition this process allows to synthesize compounds related to the compound of structure I from a common intermediate and a cheap starting material.

### SUMMARY OF THE INVENTION

The present invention provides a process for preparing the compound of the formula I from the intermediate of formula 6.

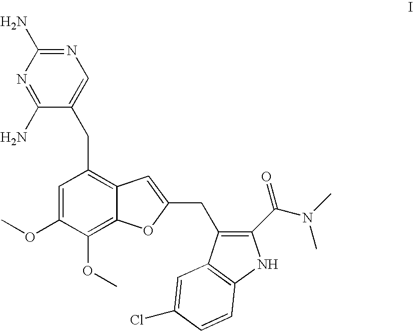

The intermediate of formula 3 is synthesized in 3 steps from a readily available starting material 1 (Scheme 1). The diamino pyrimidine substituent of 1 is selectively protected according to R. J. Griffin et al., J. Chem. Soc. Perkin Trans I, 1811 (1992) leading to compound of formula 2, which in turn is formylated to a compound of formula 3 (Scheme 1).

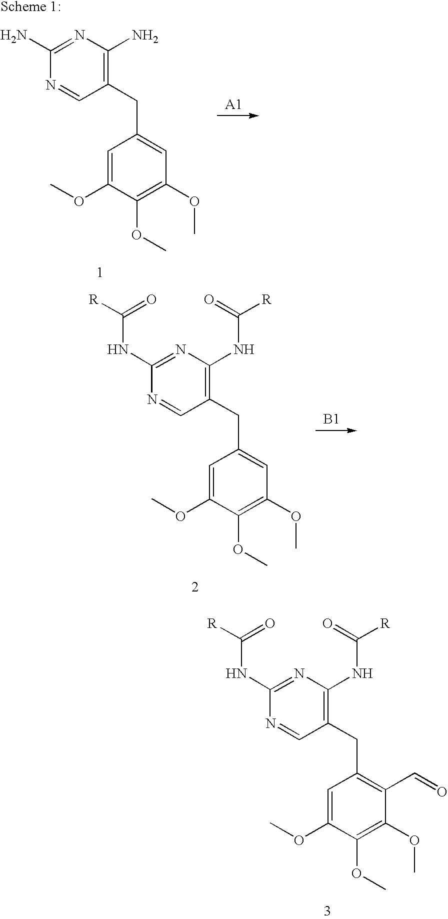

Another route of obtaining the compound of formula 3 is depicted in Scheme 2. Compound of formula 4 is protected at the diamino pyrimidine group to a compound of formula 5 followed by carbonylation of 5 to the intermediate of structure 3 (Scheme 2).

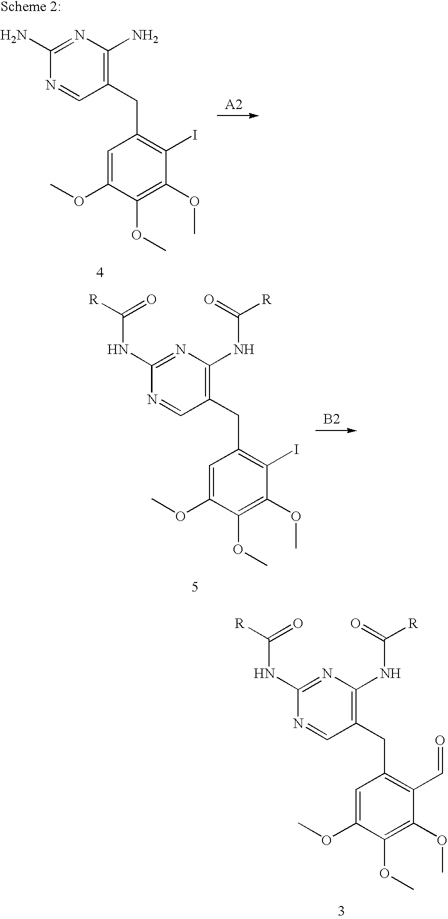

The compound of formula 3 is transformed by selective demethylation to the key intermediate of formula 6 (Scheme 3).

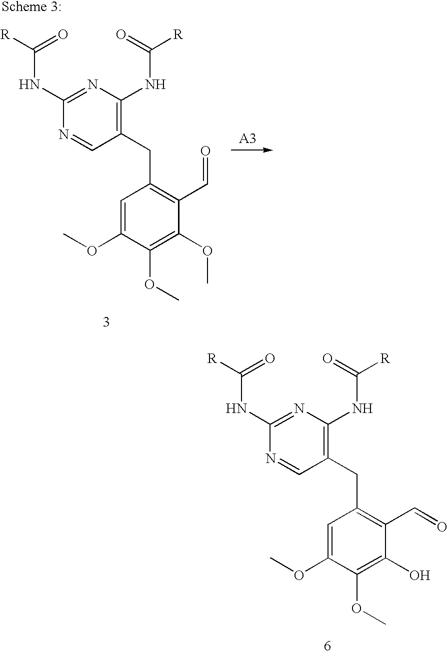

The synthesis of compound I deserves an additional intermediate of formula 10 or 12. Compounds of formulae 10/12 are synthesized from the commercially available compound of formula 7 (e.g. available from Fluka AG or Biosynth AG), which is acetylated to compound of formula 8. Compound of formula 8 was N-protected with tosyl-chloride to obtain the compound of formula 9 with subsequent bromination to compound of formula 10 or the compound of formula 8 is first brominated to compound of formula 11 followed by N-protection with di-tert-butyl dicarbonate to compound 12 (Scheme 4).

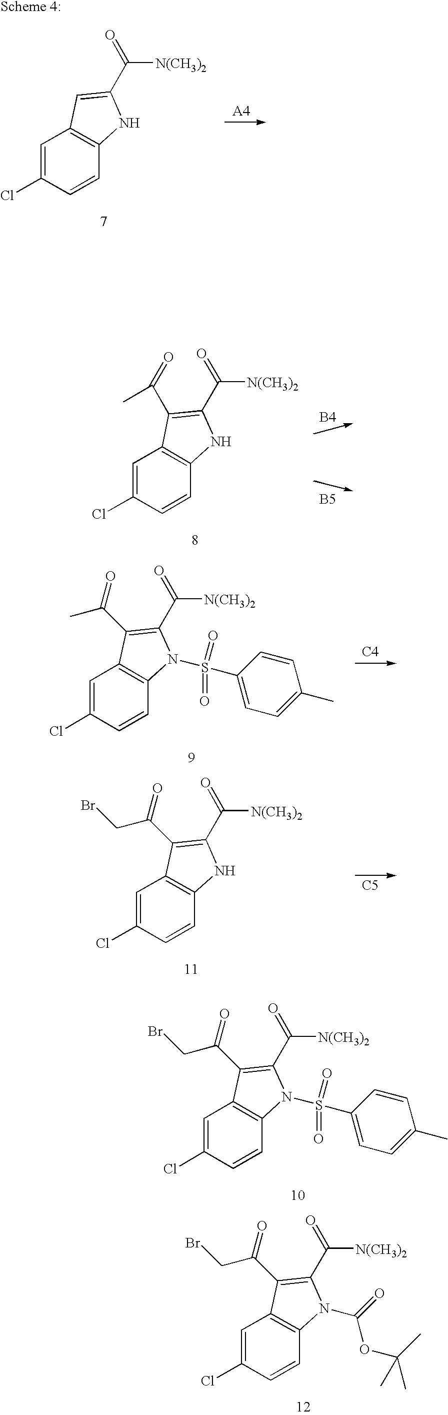

Alkylation of compound of formula 6 at the phenolic group with either bromo-ketone of formulae 10 or 12 and subsequent cyclisation leads to the furan derivatives of formulae 13 or 15 respectively. Compounds of formulae 13 and 15 are deprotected to the compound of formula 14 (Scheme 5).

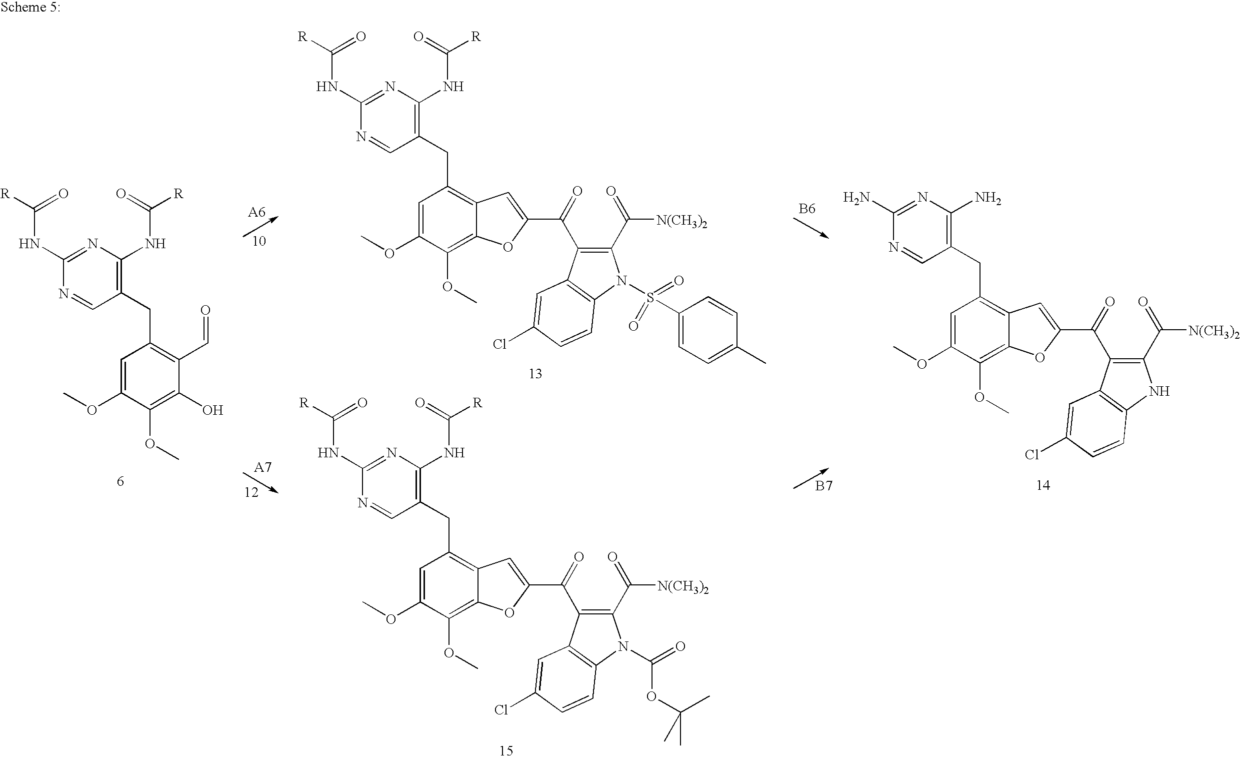

The keto carbonyl group of compounds of formulae 13 or 15 are transformed into a methylene group and compounds of formulae 16 or 17 respectively (Scheme 6) are obtained.

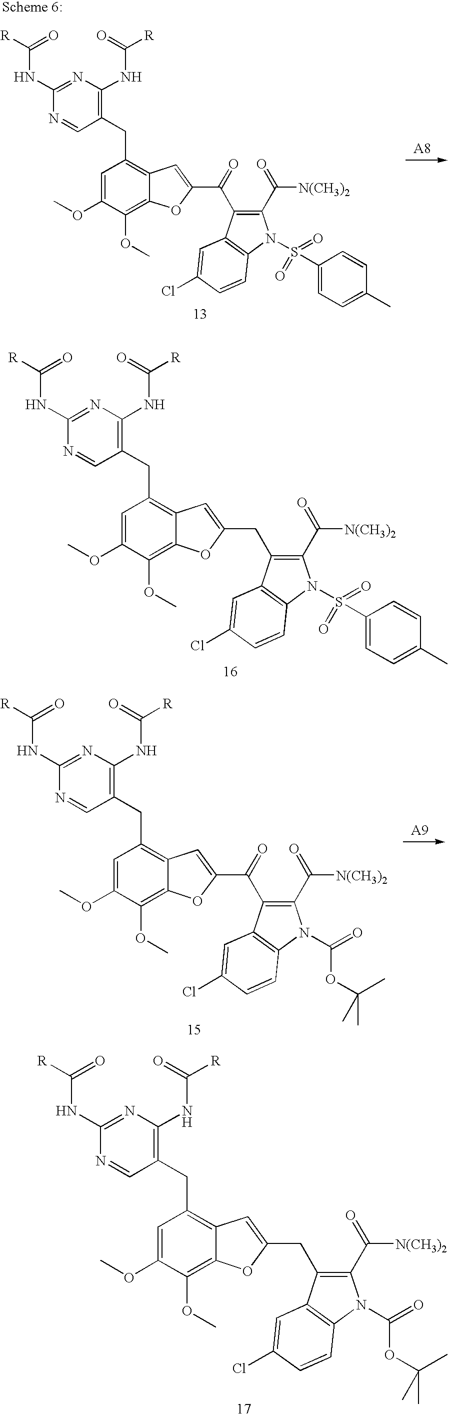

Transformation of the keto carbonyl group of compound 14 leads directly to the target compound of formula I. The reduction can also be done in two steps via the alcohol 14A (Scheme 7).

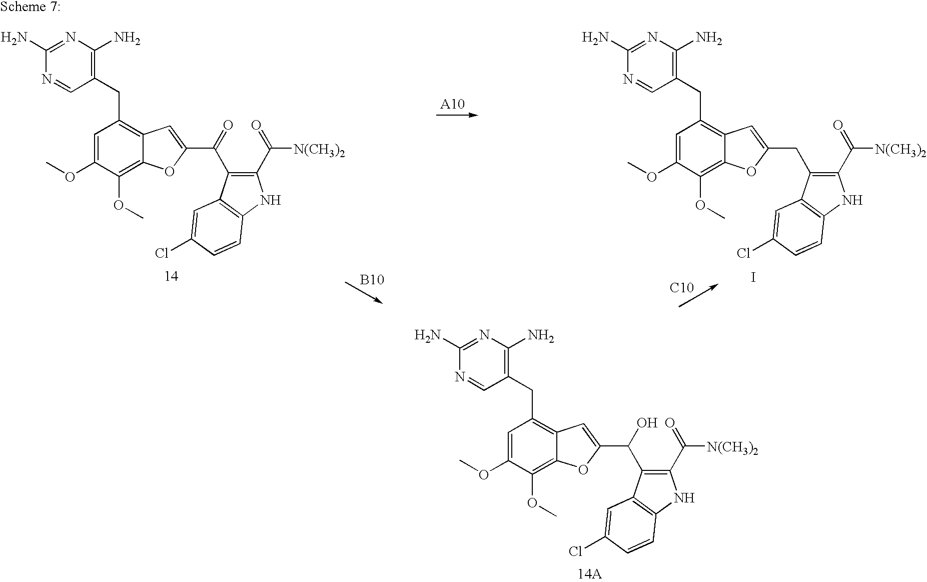

Deprotection of compounds of formulae 16 and 17 lead also directly to the compound of formula I (Scheme 8).

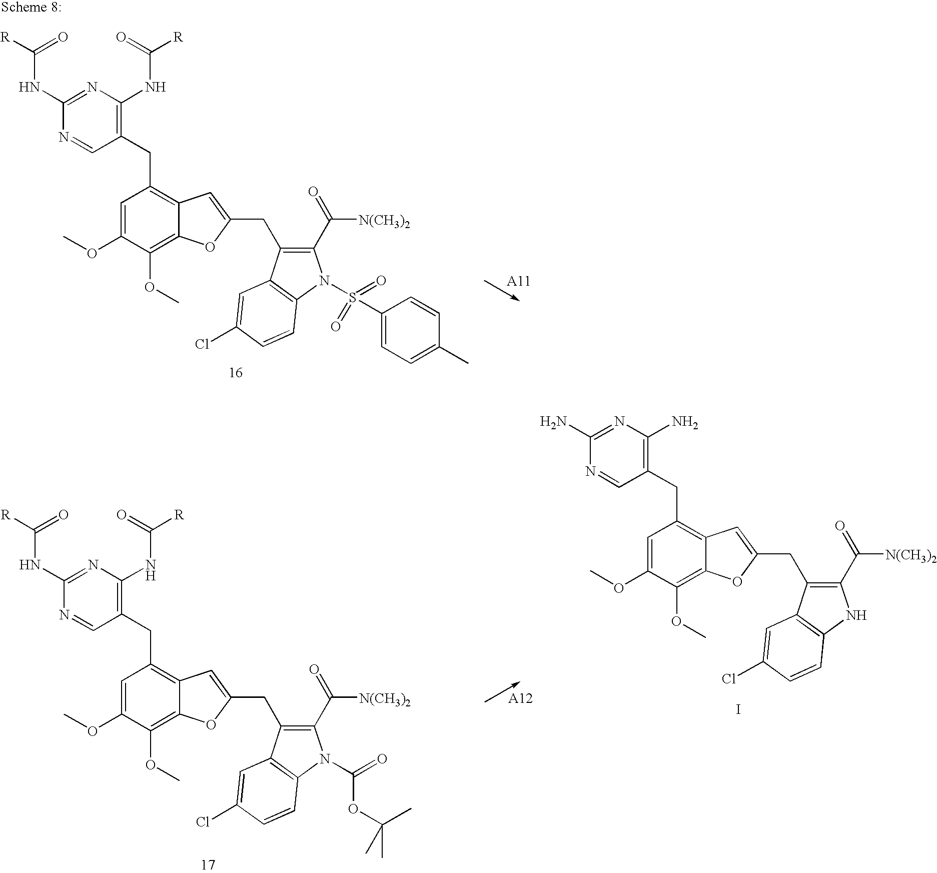

The compound of formula I is basic in nature and can be, if desired, transformed with an acid into pharmaceutically acceptable salts. Suitable acids are, e.g. hydrochloric acid, maleic acid, succinic acid, L(+)-lactic acid, DL-lactic acid, glycolic acid, 1-hydroxy-naphthalene-2-carboxylic acid, tartaric acid, citric acid, methane sulfonic acid. Most preferred are carboxylic acids.

### DETAILED DESCRIPTION OF THE INVENTION

The process of the present invention provides many advantages and improvements over the current process of synthesizing compound of formula I as described in the patent application PCT/EP 2004/007482. The corresponding starting materials of formulae 1 and 7 are commercially available in bulk quantities.

The central intermediate of formula 6 to prepare the compound of formula I may be prepared following the reaction sequences depicted in Schemes 1 to 3. The protection Al of trimethoprim 1 can be done by heating compound of formula I with acid anhydrides, e.g. acetic anhydride, isobutyric acid anhydride or pivaloyl acid anhydride in an inert, high boiling solvent like toluene, p-xylene or in plain acid anhydride up to about 120° C. to 160° C. The formulation B1 of the protected trimethoprim 2 can be achieved in an inert solvent, e.g. dichloromethane, dichloroethane, preferably dichloromethane with dichloromethyl-methyl ether and a Lewis acid, e.g. tin tetrachloride at 0° C. to −30° C., preferably at −10° C. Alternatively, compound of formula 3 can also be synthesized via protection A2 of compound 4 with acid anhydrides, e.g. acetic anhydride, methyl-propionic acid anhydride or pivaloyl acid anhydride in an inert, high boiling solvent like toluene, p-xylene or in plain acid anhydride, preferably methyl-propionic acid anhydride up to about 120° C. to 160° C. Carbonylation B2 of compound of formula 5 can be effected in an inert atmosphere and solvent, e.g. tetrahydrofuran, with palladium tetrakis as catalyst, carbon monoxide and tri-butyl tin-hydride at 60° C. to 80° C. The selective demethylation A3 can be done in an inert solvent, e.g. dichloromethane, acetonitrile, in combination with a Lewis acid like aluminium trichloride, boron trichloride, boron tribromide, manganese dichloride, manganese diiodide, preferably aluminium trichloride and a nucleophile, e.g. sodium iodide, dimethyl sulfide, diethyl sulfide, tetrahydrothiophene, preferably sodium iodide at room temperature up to 40° C.

The convergent synthesis strategy of compound of formula I deserves an additional intermediate of formulae 10 or 12. The starting material of formula 7 is acetylated (A4) with acetyl chloride and a Lewis acid like aluminium trichloride or tin tetra-chloride at ambient temperature to the intermediate of formula 8. Compound of formula 8 can be converted by protecting first (B4) the nitrogen with a sulfonyl chloride, e.g. benzyl- or p-toluene-sulfonyl chloride with a base like triethylamine, pyridine in an inert solvent at room temperature to the compound of formula 9 followed by bromination (C4) with e.g. bromine, N-bromosuccinimid, copper (II) bromide, preferably bromine of the acetyl group in dioxane to compound of formula 10, or bromination first with e.g. bromine, N-bromosuccinimid, preferably bromine of 8 (B5) in an inert solvent like dioxane at room temperature to compound of formula 11 and subsequent protection (C5) with di-tert-butyl dicarbonate and a pyridine base, e.g. 2,6-dimethyl-pyridine with 4-dimethylamino-pyridine as catalyst to compound of formula 12 at ambient temperature.

Alkylation of compound of formula 6 with either compound of formulae 10 (A6) or 12 (A7) and subsequent cyclisation in an inert solvent, e.g. dimethyl formamide, tetrahydrofuran, preferably tetrahydrofuran with a base like sodium carbonate, potassium tertiary butoxide, preferably potassium tertiary butoxide at ambient temperature up to 40° C., preferably at room temperature leads to compound of formulae 13 respectively 15. Both compounds of formulae 13 and 15 are deprotected (B6, B7) in a mixture of solvents, e.g. tetrahydrofuran, methyl alcohol, preferably tetrahydrofuran and water with a strong base like sodium or potassium hydroxide, preferably sodium hydroxide at 40° C. to 80° C. preferably at 50° C. to the compound of formula 14.

The reduction A8 respectively A9 of the ketone functional group of compound of formulae 13 and 15 respectively can be achieved with trimethyl-silane in trifluoro acetic acid at ambient temperature and leads to the compounds of structures 16 and 17 as shown in Scheme 6.

Reduction A10 of the keto function of compound of formula 14 can be done with a reducing agent, e.g. sodium borohydride, sodium cyanoborohydride, zinc borohydride, sodium acetoxyborohydride, preferably sodium cyanoborohydride, sodium borohydride or zinc borohydride in an organic solvent like methanol, isopropanol, tetrahydrofuran, dimethoxyethane or a mixture thereof, preferably isopropanol or tetrahydrofuran at temperature in the range of −20° C. up to 70° C. depending on the reducing agent leading to the target compound I. Or, via a two step reduction B10 and C10 of compound of formula 14 to the intermediate alcohol 14A with sodium borohydride at −20° C. or with a ruthenium catalysts at room temperature and subsequent reduction to the final compound I with sodium borohydride at 0° C. with boron trifluoride or trifluoroacetic acid as a catalyst.

Deprotection A11 and A12 of compounds of formula 16 and 17 can be achieved in a mixture of organic solvents, e.g. tetrahydrofuran, methyl alcohol, preferably tetrahydrofuran and water with a strong base like sodium or potassium hydroxide, preferably sodium hydroxide at 40° C. to 80° C. preferably at 50° C. leading to the target compound I.

The compounds of formulae 2, 3, 5, 6, 8 to 17 are novel and are also objects of the invention. They can be prepared according to the reaction sequences elucidated in Schemes 1 to 8. The preparation of compounds outlined in Schemes 1 to 8 are, moreover, described in more detail in the examples.

As already mentioned, the compound of formula I or their pharmaceutically acceptable salts have valuable antibacterial properties. These compounds are active against a large number of pathogenic microorganisms such as e.g. S. aureus, P. carinii etc. by virtue of their activity in inhibiting bacterial dihydrofolate reductase (DHFR). The activity of compound I is described in patent application PCT/EP 2004/007482.

Additional objects, advantages, and novel features of this invention will become apparent to those skilled in the art upon examination of the following examples, which are not intended to be limiting the scope of the invention.

The following examples illustrate the invention in more detail. Examples 1 to 11 describe the preparation of compound 6, while examples 12 to 16 describe the preparation of the compound of formulae 10 and 12, and examples 17 to 32 describe the condensation of the compound of formula 6 with those of formulae 10 or 12 to the end product of formula I.

### EXAMPLES

Compound of formula 4 can be prepared e.g. according to M. Calas et al., Eur. J. Med. Chem. Chim. Ther., 17 (6), 497 (1982). Compound 7 can be prepared in analogy to e.g. W. B. Wright et al., J. Med. Chem., 11 (6), 1164 (1968).

All other reagents and solvents are readily commercially available, for example from Fluka or equivalent commercial suppliers. The temperatures are given in degrees Celsius.

### LCMS System

HPLC Column 01: Reverse Phase, Waters C₁₈ 3.5 μm 4.6×75 mm column Gradient 01:

| Time | | | |
|---|---|---|---|
| Min. | Flow mL | % A water/10 mM Formic acid | % B Acetonitrile |
| 0.00 | 1.2 | 95 | 5 |
| 5.00 | 1.2 | 50 | 50 |
| 6.00 | 1.2 | 5 | 95 |
| 9.00 | 1.2 | 5 | 95 |
| 11.00 | 1.2 | 95 | 5 |
| 12.00 | 1.2 | 95 | 5 |

HPLC Column 02: Reverse Phase, Waters C₁₈ 3.5 μm 3×20 mm column Gradient 02:

| Time | | | |
|---|---|---|---|
| Min. | Flow mL | % A water/10 mM Formic acid | % B Acetonitrile |
| 0.00 | 0.7 | 95 | 5 |
| 3.00 | 0.7 | 5 | 95 |
| 3.50 | 0.7 | 5 | 95 |
| 3.60 | 0.7 | 95 | 5 |
| 4.50 | 0.7 | 95 | 5 |

Solvent A: 10 mM Formic acid (Formic acid 377 μl) was added to HPLC grade water (1 L, Millipore filtered)
Solvent B: Acetonitrile HPLC grade (Biosolve Ltd)

### Wavelength: 210 nm to 400 nm.

HPLC Apparatus Type: Finnigan StartSystem SCN1000, Finnigan Photodiode array detector(PDA) UV6000LP
MS Apparatus Type: Finnigan LCQ (ION TRAP), lonisation mode ESI

Abbreviations

| | |
|---|---|
| AcOEt | Acetic acid ethylester |
| AlCl₃ | Aluminium trichloride |
| CH₃CN | Acetonitrile |
| DCM | Dichloromethane |
| DMAP | 4-Dimethylamino pyridine |
| DMF | Dimethylformamide |
| DMSO | Dimethyl sulfoxide |
| eq. | Equivalent |
| iPAc | Isopropyl acetate |
| iPrOH | Isopropyl alcohol |
| LCMS | High pressure liquid chromatography with MS detection |
| MgSO₄ | Manganese sulfate |
| MOM-Cl | Chloromethyl-methylether |
| mp | Melting point |
| NaHCO₃ | Sodium hydrogen carbonate |
| RT | Room temperature |
| Rₜ (01) | Retention time column/gradient 01 |
| Rₜ (02) | Retention time column/gradient 02 |
| TBME | Tertiary butyl methyl ether |
| tBuOK | Potassium tertiary butoxide |
| TFA | Trifluoroacetic acid |
| THF | Tetrahydrofuran |

### Example 1

This example illustrates the preparation of N-[4-(2,2-Dimethyl-propionylamino)-5-(3,4,5-trimethoxy-benzyl)-pyrimidin-2-yl]-2,2-dimethyl-propionamide 2 (R=C(CH₃)₃) (step A1).

A solution of trimethoprim (5 g, 17.24 mmol) in pivalic anhydride (8.74 mL, 43.10 mmol, 2.5 eq.) was heated during 2 h at 150° C. under argon. Hot AcOEt was added, and the organic layers were washed with aqueous NaHCO₃ 10%, water and brine. The org. layers were then dried over MgSO₄, filtered and evaporated. It was then recrystallized from TBME to give 3.02 g of compound 2 (R=C(CH₃)₃).

### ¹H-NMR (CDCl₃, 400 MHz) δ: 8.35 (s, 1 H), 8.21 (br s, 1 H), 7.65 (br s, 1 H), 6.30 (s, 2 H), 3.86 (s, 2 H), 3.79 (s, 3 H), 3.77 (s, 6 H), 1.31 (s, 9 H), 1.12 (s, 9 H). mp: 130-133° C.

### Example 2

This example illustrates the preparation of N-[4-isobutyrylamino-5-(3,4,5-trimethoxy-benzyl)-pyrimidin-2-yl]-isobutyramide 2 (R=CH(CH₃)₂) (step A1).

A solution of trimethoprim (50 g, 172.4 mmol) in isobutyric anhydride (100 g, 105 mL, 632 mmol, 3.6 eq.) was heated during 2 h at 150° C. under argon. The warm solution was poured into 1 L of cyclohexane from where it slowly crystallized. The product was filtered off and was washed thoroughly with cyclohexane (2×200 mL) to give 70 g of compound 2 (R=CH(CH₃)₂).

¹H-NMR (D₆-DMSO, 400 MHz) δ: 10.42 (s, 1H, NH); 10.15 (s, 1H, NH); 8.41 (s, 1H, pyrimidine); 6.41 (s, 2H, PhH); 3.81 (s, 2H, CH₂); 3.70 (s, 6H, 2×OCH₃); 3.59 (s, 3H, OCH₃); 2.72-2.85 (m, 2H, CH); 1.06 (d, 6H, J=6.6 Hz, 2×CH₃), 1.01 (d, 6H, J=6.6 Hz, 2×CH₃. mp: 153-154° C. Rₜ (02)=1.65 minutes.

### Example 3

This example illustrates the preparation of N-[4-isobutyrylamino-5-(3,4,5-trimethoxy-benzyl)-pyrimidin-2-yl]-isobutyramide 2 (R=CH(CH₃)₂) (step A1).

A solution of trimethoprim (50 g, 172.4 mmol) in isobutyric anhydride (62 g, 65.5 mL, 392 mmol, 2.3 eq.) was heated during 2 h at 150° C. under Ar and stirred with a mechanical stirrer. The solution was cooled to 130° C. and 200 ml toluene was added (clear solution), then 1000 ml TBME was slowly added (after 500 ml crystallization started) under vigorous stirring. The thick crystal cake was stirred for 1 hour at 100° C. external temperature. Then the slurry was cooled to RT and stirred for 2 hours. Finally the slurry was cooled to 10° C. and stirred for 2 hours. The crystals were filtered and washed with 3 times 90 ml TBME to remove residual isobutyric acid and anhydride. The crystals were dried at HV/70° C. for 8 hours to give 70 g of compound 2 (R=CH(CH₃)₂).

¹H-NMR (D₆-DMSO, 400 MHz) δ: 10.42 (s, 1H, NH); 10.15 (s, 1H, NH); 8.41 (s, 1H, pyrimidine); 6.41 (s, 2H, PhH); 3.81 (s, 2H, CH₂); 3.70 (s, 6H, 2×OCH₃); 3.59 (s, 3H, OCH₃); 2.72-2.85 (m, 2H, CH); 1.06 (d, 6H, J=6.6 Hz, 2×CH₃), 1.01 (d, 6H, J=6.6 Hz, 2×CH₃. mp: 153-154° C. Rₜ (02)=1.65 minutes.

### Example 4

This example illustrates the preparation of N-[4-(2,2-Dimethyl-propionylamino)-5-(2-formyl-3,4,5-trimethoxy-benzyl)-pyrimidin-2-yl]-2,2-dimethyl-propionamid 3 (R=C(CH₃)₃) (step B1).

To a solution of 2 (1 g, 2.18 mmol, R=C(CH₃)₃) in DCM (5 mL), dichloromethyl methyl ether (0.58 mL, 6.54 mmol) was added. The solution was cooled to −30° C. before slowly adding stannic chloride (0.285 mL, 2.18 mmol). The mixture was stirred at a temperature between −10° C. and −5° C. At 0° C., the reaction mixture was poured into a solution of 1-N K₃PO₄. The mixture (pH 7-8) was then vigorously stirred during 15 minutes, extracted twice with AcOEt. The organic layers were washed with water and brine, dried over MgSO₄, filtered and evaporated. The crude product was purified by flash chromatography on silica gel (AcOEt/Cyclohexane 7/3) to give 709 mg of compound 3 (R=C(CH₃)₃).

### ¹H-NMR (CDCl₃, 400 MHz) δ: 10.23 (s, 1 H), 8.44 (s, 1 H), 8.12 (s, 1 H), 8.09 (s, 1 H), 6.59 (s, 1 H), 4.10 (s, 2 H), 3.96 (s, 3 H), 3.89 (s, 3 H), 3.85 (s, 3 H), 1.29 (s, 9 H), 1.27 (s, 9 H).

### mp: 124-126° C.

### Example 5

This example illustrates the preparation of N-[5-(2-Formyl-3,4,5-trimethoxy-benzyl)-4-isobutyrylamino-pyrimidin-2-yl]-isobutyramide 3 (R=CH(CH₃)₂) (step B1).

To a solution of 2 (70 g, 162 mmol, R=CH(CH₃)₂) in DCM (500 mL), dichloromethyl methyl ether (30 mL, 325 mmol) was added. The solution was cooled to −10° C. before slowly adding stannic chloride (35 mL, 300 mmol). The mixture was stirred at a temperature between −10° C. and −5° C. At first a gummy precipitate was formed (mechanical stirrer required). After 1 h stirring at −5° C. this was transformed into a “homogeneous” suspension.

At 0° C., the reaction mixture is poured into a solution of 300 mL 1 N K₃PO₄ and 200 mL 1 M Na/K-tartrate while cooling with an ice bath. The mixture (pH was adjusted with 4N NaOH solution to 7-8) was then stirred for 15 minutes until complete hydrolysis, and then extracted with DCM (300 mL) together with AcOEt (500 mL). The organic layer was washed with 0.1 N HCl solution (2×200 mL) and brine (2×300 mL), dried over MgSO₄, filtered and evaporated. The product precipitated while concentration to about half of the initial volume, cyclohexane (200 mL) was added to further precipitate the product which was then filtered off to give 50 g of compound 3 (R=CH(CH₃)₂).

¹H-NMR (D₆-DMSO, 400 MHz) δ: (s, 1H, NH); 10.27 (s, 1H, NH); 10.17 (s, 1H, CHO); 8.00 (s, 1H, pyrimidine); 6.72 (s, 1H, PhH); 4.05 (s, 2H, CH₂); 3.90 (s, 3H, OCH₃); 3.85 (s, 3H, OCH₃); 3.77 (s, 3H, OCH₃); 2.75-2.85 (m, 2H, CH); 1.05 (d, 6H, J=6.6 Hz, 2×CH₃), 1.01 (d, 6H, J=6.6 Hz, 2×CH₃). mp: 162-163° C. Rₜ (02)=1.85 minutes.

### Example 6

This example illustrates the preparation of N-[5-(2-Formyl-3,4,5-trimethoxy-benzyl)-4-isobutyrylamino-pyrimidin-2-yl]-isobutyramide 3 (R=CH(CH₃)₂) (step B1).

Dichloromethyl-methyl ether (4.3 mL, 46.4 mmol, 2 eq.) was dissolved in DCM (30 mL) and cooled to −15° C. to −20° C. in a reaction vessel with mechanical stirrer. To this solution stannic chloride (5 mL, 42.8 mmol, 1.8 eq.) was added within 15 minutes. The clear solution was stirred at −18° C. for 30 minutes. Then a solution of 2 (10 g, 23.2 mmol, crystallized from toluene and TBME) in DCM (40 mL) was continuously added during 60 minutes, a yellow solid separated from the beginning and resulted in a thick slurry (green/yellow). Then the slurry was stirred at −15° C. for two hours, at −10° C. for one hour and 30 minutes at −5° C. Then 40 mL DCM was added at −5° C. and the separated crystals at the top of the solvent layer were removed with vigorous stirring for 15 minutes. The thin slurry was transferred into a well-stirred mixture of 35 g Na₂CO₃ (with one crystal water) dissolved in 100 mL water and 35 mL DCM at 10° C. The mixture was stirred for 15 minutes at RT and then transferred back to the reaction vessel to finish the workup continuing the stirring at RT. After vigorous stirring for 30 minutes at RT the layers were separated and the organic phase washed twice with a mixture of 30 mL saturated NaCl, 5 ml saturated Na₂CO₃ and 40 mL water (several shakings are necessary, the water phases should show a pH of 7 to 8). The milky water phases were washed with 50 mL DCM and 30 mL DCM separately.

The organic layers were dried over MgSO₄, filtered and evaporated. The crude white crystalline product was crystallized from DCM and TBME.

With 10.67 g crude material a slurry was made in 25 mL DCM at 44° C. under stirring for 30 minutes and 100 mL TBME were slowly added and the slurry stirred for 30 minutes at 44° C. and then cooled to RT for 6 hours under stirring. The crystals were filtered and washed with 40 mL TBME, dried at high vacuum/RT for 6 hours to give 9.6 g of compound 3 (R=CH(CH₃)₂).

¹H-NMR (D₆-DMSO, 400 MHz) δ: (s, 1H, NH); 10.27 (s, 1H, NH); 10.17 (s, 1H, CHO); 8.00 (s, 1H, pyrimidine); 6.72 (s, 1H, PhH); 4.05 (s, 2H, CH₂); 3.90 (s, 3H, OCH₃); 3.85 (s, 3H, OCH₃); 3.77 (s, 3H, OCH₃); 2.75-2.85 (m, 2H, CH); 1.05 (d, 6H, J=6.6 Hz, 2×CH₃), 1.01 (d, 6H, J=6.6 Hz, 2×CH₃). mp: 162-163° C. Rₜ (02)=1.85 minutes.

### Example 7

This example illustrates the preparation of N-[4-(2,2-Dimethyl-propionylamino)-5-(2-iodo-3,4,5-trimethoxy-benzyl)-pyrimidin-2-yl]-2,2-dimethyl-propionamide 5 (R−C(CH₃)₃) (step A2).

To a solution 4 (5 g, 9.2 mmol, R=C(CH₃)₃) in pivalic anhydride (4.1 mL, 20.24 mmol) was added pyridine (1.65 mL, 20.24 mmol). The mixture was heated at 120° C. during 12 h. HCl 0.25 N (25 mL) was added and the mixture was extracted twice with AcOEt. The organic layers were washed subsequently with water, NaHCO₃ 10%, then water and brine, dried over MgSO₄, filtered and evaporated to dryness. The final compound was obtained by flash chromatography on silica gel (AcOEt/Cyclohexane 1/1) to give 2.5 g of compound 5 (R=C(CH₃)₃). UV: 238 (282) nm.

### Example 8

This example illustrates the preparation of N-[4-(2,2-Dimethyl-propionylamino)-5-(2-formyl-3,4,5-trimethoxy-benzyl)-pyrimidin-2-yl]-2,2-dimethyl-propionamid 3 (R=C(CH₃)₃) (step B2).

(1 g, 1.71 mmol, R=C(CH₃)₃) was dissolved in THF (10 mL) and the mixture was degassed with argon. Palladium tetrakis was then added (49.4 mg, 4 mol %). The mixture was heated to 70° C. and a slow stream of carbon monoxide gas was started. Bu₃SnH (476 μL, 1.05 eq.) in 5 mL THF was slowly added over a period of 2.5 h. After 12 h at 70° C., compound 3 (R=C(CH₃)₃) was isolated by flash chromatography. The analytical data were comparable to the compound of Example 4.

### Example 9

This example illustrates the preparation of N-[4-(2,2-Dimethyl-propionylamino)-5-(2-formyl-3-hydroxy-4,5-dimethoxy-benzyl)-pyrimidin-2-yl]-2,2-dimethyl-propionamide 6 (R=C(CH₃)₃) (step A3).

Under argon, 3 (2 g, 4.11 mmol, R=C(CH₃)₃) was dissolved in DCM (10 mL). AICI₃ (823 mg, 6.17 mmol) was added to the mixture at 0° C. Stirring was continued at RT during 10 minutes until complete dissolution of AlCl₃. Sodium iodide (616 mg, 4.11 mmol) was added, and after 30 minutes, 0.5 mL of acetonitrile was added. The reaction was checked by LCMS, and 0.5 mL acetonitrile was added to complete the reaction. The reaction mixture was then poured into 1 N K₃PO₄/DCM biphasic solution. The two phases were separated. The aqueous layers were extracted twice with AcOEt and the organic layers were washed with water and brine, then dried on MgSO₄, filtered and evaporated. Compound 6 (R=C(CH₃)₃) was obtained after purification on a column chromatography on silica gel eluting with AcOEt/cyclohexane 6/4 (1.2 g).

### ¹H-NMR (CDCl₃, 400 MHz) δ: 12.07 (s, 1 H), 9.81 (s, 1 H), 8.94 (s, 1 H), 8.26 (s, 1 H), 7.95 (s, 1 H), 6.33 (s, 1 H), 4.07 (s, 2 H), 3.84 (s, 3 H), 3.80 (s, 3 H), 1.22 (s, 9 H), 1.20 (s, 9 H). mp: 110-112° C.

### Example 10

This example illustrates the preparation of N-[5-(2-Formyl-3-hydroxy-4,5-dimethoxy-benzyl)-4-isobutyrylamino-pyrimidin-2-yl]-isobutyramide 6 (R=CH(CH₃)₂) (step A3).

Under argon, 3 (4 g, 8.7 mmol, 1 eq., R=CH(CH₃)₂) were dissolved in DCM (36 mL). Aluminium trichloride (3.48 g, 26.1 mmol, 3 eq.) and sodium iodide (2 g, 13.3 mmol, 1.5 eq.) were added to the mixture at RT. The mixture was stirred for 20 minutes before acetonitrile (2.4 mL) was added and then warmed up to 40° C. Stirring at 40° C. was continued for 3.5 h. The mixture was cooled to RT, diluted with 75 mL DCM and quenched by adding the reaction mixture to 30 mL ice-water, then 2.5 mL of concentrated HCl was slowly added, which helped to dissolve the yellow precipitate. The organic layer was separated and the aqueous layer extracted once more with DCM (75 mL). The combined organic layers were washed with brine (50 mL), twice with sodium bicarbonate solution made from 50 mL saturated sodium bi-carbonate (NaHCO₃)+150 mL water (2×100 mL), 0.1N HCl solution (50 mL) and again brine (1×50 mL). The resulting yellowish solution was dried over MgSO₄ and concentrated. The oily residue was crystallized from ethyl acetate (6 mL) and dichloroethane (2.4 mL) by first warming to 50° C., then cooling to 4° C. After filtration the mother liquor was concentrated to halve and stored at 4° C. to give a second crop of crystals. In total 2.48 g of compound 6 (R=CH(CH₃)₂) were isolated.

¹H-NMR (CDCl₃, 400 MHz) δ: 11.95 (brs, 1H, PhOH); 9.9 (s, CHO, 1H), 8.04 (s, pyrimidine, 1H); 6.44 (s, ArH, 1H), 4.15 (s, CH₂, 2H), 3.93 (s, OCH₃, 3H); 3.90 (s, OCH₃, 3H); 2.7-2.8 (m, CH, 2H); 1.2-1.25 (m, CH₃, 12H).

### Example 11

This example illustrates the preparation of N-[5-(2-Formyl-3-hydroxy-4,5-dimethoxy-benzyl)-4-isobutyrylamino-pyrimidin-2-yl]-isobutyramide 6 (R=CH(CH₃)₂) (step A3).

Under argon, 3 (3 g, 6.54 mmol) was dissolved in DCM (28 mL) at RT (15 minutes). The solution was cooled to 0° C. during 30 minutes. AlCl₃ (2.07 g, 15.52 mmol, 2.3 eq.) was added to the cooled solution at once. The colour of the solution changed from yellow to dark yellow within 30 minutes and the AlCl₃ dissolved while stirring for 30 minutes at 0° C. Then NaI (1.5 g, 10 mmol, 1.5 eq.) was added and the mixture warmed up to 30° C. After 10 minutes stirring acetonitrile (1.6 mL) was slowly added. After 2 hours stirring at 30° C. additional 0.2 mL acetonitrile was added. After 4 hours stirring at 30° C. the reaction temperature was raised to 35° C. for 1 h, during this time crystals separated. The slurry was cooled to RT and then poured into a well stirred mixture of 20 mL DCM and 30 mL water containing 2 mL concentrated HCl (cooled to 10° C.). After stirring for 10 minutes the clear yellow mixture was poured back to the reaction vessel and the stirring was continued until all the residues were dissolved (about 30 minutes). The organic phase was separated and washed with 25 mL of a mixture of 10 mL 1-N HCl and 15 mL water and 25 mL of a mixture of 10 ml saturated NaCl and 15 ml water, the water layers were extracted with 2 times 20 mL DCM. The combined organic solution was dried with MgSO₄, filtered and evaporated. The yellow crystalline residue (2.62 g) was crystallized from DCM/TBME to give 2.48 g of compound 6 (R=CH(CH₃)₂).

¹H-NMR (CDCl₃, 400 MHz) δ: 11.95 (brs, 1H, PhOH); 9.9 (s, CHO, 1H), 8.04 (s, pyrimidine, 1H); 6.44 (s, ArH, 1H), 4.15 (s, CH₂, 2H), 3.93 (s, OCH₃, 3H); 3.90 (s, OCH₃, 3H); 2.7-2.8 (m, CH, 2H); 1.2-1.25 (m, CH₃, 12H).

### Example 12

This example illustrates the preparation of 3-Acetyl-5-chloro-1H-indole-2-carboxylic acid dimethylamide 8 (step A4).

Aluminium trichloride (36 g, 270 mmol) was added slowly to a suspension of 7 (30 g, 135mmol) in DCM (675 mL) at 0° C. under Ar. The reaction mixture was stirred for 30 minutes and acetyl chloride (9.6 mL, 135 mmol) was added dropwise at 0° C. The gold yellow reaction mixture was stirred for an additional 1 hour, until the reaction was completed (verification by LC-MS).

The reaction mixture is then poured on ice (250 mL). The pH was adjusted to pH 4.5 by addition of 4 N NaOH solution (80 mL). The phases were separated and the aqueous layer was extracted with DCM (2×200 mL). All collected organic layers were then washed with water and brine, then dried over MgSO₄, filtered and evaporated. The compound 8 was obtained as a beige solid and used for the next reaction step without further purification.

¹H-NMR (CDCl₃, 400 MHz) δ: 12.6 (brs, 1 H), 8.15 (d, J=2 Hz, 1 H), 7.47 (d, J=8 Hz, 1 H), 7.27 (dd, J₁=8.6 Hz, J₂=2.6 Hz, 1 H), 3.08 (s, 3 H), 2.84 (s, 3 H), 2.37 (s, 3 H). mp: 150-155°.

### Example 13

This example illustrates the preparation of 3-Acetyl-5-chloro-1-(toluene-4-sulfonyl)- 1H-indole-2-carboxylic acid dimethylamide 9 (step B4).

40 g (151.1 mmol) of 8 were dissolved in DCM (250 mL), at RT under Ar. Then, triethylamine (36.4 mL) and tosylchloride (31.7 g, 166.2 mmol) were added and the reaction mixture was stirred overnight. The excess of tosylchloride was quenched by adding 15 mL of a solution of ammonia in water (24%) to the reaction mixture. After stirring for 10 minutes, the mixture was washed with 0.1 N NaOH solution (50 mL) and brine (50 mL). The organic phases were dried over MgSO₄. After evaporating the combined organic phase by half, 500 mL of cyclohexane were added until the product started to precipitate. The reaction mixture was again evaporated to ca. 200 mL. Then the precipitate was filtered off and washed with cyclohexane. Drying under high vacuum gave 51.7 g of a white powder.

¹H-NMR (CDCl₃, 400 MHz) 6:8.29 (d, J=2.8 Hz, 1 H), 8.03 (d, J=8.4 Hz, 2 H), 7.95 (d, J=9.2 Hz, 1 H), 7.33 (dd, J₁=10 Hz, J₂=2 Hz, 2 H), 7.28 (d, J=8.8 Hz, 1 H), 3.24 (s, 3 H), 3.03 (s, 3 H), 2.50 (s, 3 H), 2.36 (s, 3 H). mp: 96-100° C.

### Example 14

This example illustrates the preparation of 3-(2-Bromo-acetyl)-5-chloro-1-(toluene-4-sulfonyl)-1H-indole-2-carboxylicacid-dimethyl-amide 10 (step C4).

A solution of 9 (25 g, 59.7 mmol) in dioxane (250 mL) was cooled with a water bath, and then a 2 M solution of bromine (9.55g, 1 eq.) in DCM was added dropwise. The cooling of the reaction mixture was continued and the reaction monitored by HPLC. After complete addition of the bromine, the reaction mixture was evaporated to dryness. 1 L of AcOEt was added, and the organic layer was washed with saturated NaHCO₃, brine and was dried over MgSO₄. The expected compound 10 was then recrystallized from hot AcOEt (100 mL) to give 18.2 g.

¹H-NMR (CDCl₃, 400 MHz) δ: 8.28 (d, J=2 Hz, 1 H), 7.97-8.01 (m, 3 H), 7.37 (dd, J₁=9.2 Hz, J₂=2 Hz, 1 H), 7.28 (d, J=8 Hz, 2 H), 4.23-4.48 (AB system, J=12.2 Hz, 2 H), 3.26 (s, 3 H), 3.04 (s, 3 H), 2.36 (s, 3 H). mp: 142-146° C.

### Example 15

This example illustrates the preparation of 3-(2-Bromo-acetyl)-5-chloro-1H-indole-2-carboxylic acid dimethylamide 11 (step B5).

A 2 M solution of bromine (3 g, 0.5 eq.) in DCM (10 mL) was added dropwise to a solution of 8 (10 g, 37.88 mmol) in dioxane (120 mL) at RT. The mixture was stirred 30 minutes then another solution of 2 M bromine (0.25 eq.) in DCM was added. The reaction was monitored by HPLC and more bromine solution in DCM (0.1 eq.) was added to complete the reaction.

After complete addition of the bromine, the hydro bromide salt of the indole started to precipitate. The mixture was evaporated to dryness, and then AcOEt (100 mL) was added. The organic layer was washed with NaHCO₃ 2% (50 mL); water (50 mL) and brine (30 mL), dried over MgSO₄, filtered and evaporated to dryness. The expected compound was then recrystallized from hot AcOEt (150 mL) to give 9.1 g of compound 11.

¹H-NMR (CDCl₃, 400 MHz) δ: 12.68 (brs, 1 H), 8.09 (d, J=2 Hz, 1H), 7.48 (d, J=8.4 Hz, 1 H), 7.29 (dd, J₁=8.4 Hz, J₂=2 Hz, 1 H), 4.46 (s, 2 H), 3.07 (s, 3 H), 2.82 (s, 3 H). mp: 165-170° C. (with decomposition).

### Example 16

This example illustrates the preparation of 3-(2-Bromo-acetyl)-5-chloro-2-dimethylcarbamoyl-indole-1-carboxylic acid tert-butyl ester 12 (step C5).

To a solution of 11 (11 g, 32 mmol) in a mixture of THF/CH₃CN (50 mL, 1/1), 2,6-lutidine (4.47 mL, 38.4 mmol) was added, followed by (Boc)₂O (9.07 g, 41.6 mmol). The starting material was not fully dissolved. When DMAP (391 mg, 3.2 mmol) was added, the insoluble compound solubilizes quickly and the reaction turns slowly to yellow/orange. After 15 minutes, the reaction was already finished. The mixture was evaporated to dryness, then AcOEt (300 mL) was added and the organic layers were washed three times with HCl 0.1 N (50 mL), water (100 mL) and brine (50 mL). The org. layers were dried over MgSO₄, filtered and evaporated. The expected compound was recrystallized from AcOEt (or cyclohexane/AcOEt 8/2: 100 mL) to give 9.9 g of compound 12.

¹H-NMR (CDCl₃, 400 MHz) δ: 8.16 (d, J=9.2 Hz, 2 H), 7.51-7.48 (dd, J₁=9.2 Hz, J₂=2.8 Hz, 1 H), AB system [4.53 (d, J=17.6 Hz, 1 H), 4.44 (d, J=17.6 Hz, 1 H)], 3.04 (s, 3 H), 2.85 (s, 3 H), 1.56 (s, 9 H). mp: 110-115° C.

### Example 17

This example illustrates the preparation of 3-[4-(2,4-Bis-isobutyrylamino-pyrimidin-5-ylmethyl)-6,7-dimethoxy-benzofuran-2-carbonyl]-5-chloro-1-(toluene-4-sulfonyl)-1H-indole-2-carboxylic acid dimethylamide 13 (R=CH(CH₃)₂) (step A6).

A filtered solution of tBuOK (1.14 g, 0.95 eq.) in THF (10 mL) was added to a solution of 6 (5 g, 11.26 mmol, R=CH(CH₃)₂) in THF (40 mL). The mixture was stirred 30 minutes at RT before adding slowly 10 (5.35 g, 10.725 mmol). After 1 h, more tBuOK (184 mg, 0.15 eq.) was added. One hour later, the reaction was finished. The mixture was poured into a solution of HCl 0.05 N, extracted three times with AcOEt. The organic layers were washed with water and brine, then dried over MgSO₄, filtered and evaporated to dryness. Compound 13 (R=CH(CH₃)₂) was used for the next reaction without any further purification (step B6). Rₜ (02)=2.75 minutes.

### Example 18

This example illustrates the preparation of 3-{4-[2,4-Bis-(2,2-dimethyl-propionyl-amino)-pyrimidin-5-ylmethyl]-6,7-dimethoxy-benzofuran-2-ylmethyl}-5-chloro-1-(toluene-4-sulfonyl)-1H-indole-2-carboxylic acid dimethylamide 13 (R=C(CH₃)₃) (step A6).

A solution of 10 (1.03 g, 2.08 mmol) in 8 mL DMF was cooled to 0° C. and a solution of 6 (0.98 g, 1 eq., R=C(CH₃)₃) was added. Then potassium carbonate (0.86 g, 3 eq.) was added and the solution warmed to RT. After 16 h stirring at RT EtOAc (150 mL) was added and washed with 0.1 N HCl (100 mL), twice with water (200 mL) and with brine. The aqueous layers were washed with additional EtOAc (100 mL). The organic layers were dried over MgSO₄, filtered and evaporated to dryness. The crude material was purified by flash chromatography on silica gel (AcOEt/cyclohexane 6/4) to give 0.93 g of compound 13 (R=C(CH₃)₃).

### Example 19

This example illustrates the preparation of 5-Chloro-3-[4-(2,4-diamino-pyrimidin-5-yl-methyl)-6,7-dimethoxy-benzofuran-2-carbonyl]-1 H-indole-2-carboxylic acid dimethyl-amide, mesylate salt 14 (step B6).

The crude materiel 13 (R=CH(CH₃)₂) was dissolved in THF/H₂O (30 mL, 3/1), NaOH (2.145 g, 53.625 mmol) was added and the mixture was heated during 4 h at 50° C. The mixture was then poured into brine and extracted 3 times with AcOEt. The organic layers were washed with brine, dried over MgSO₄, filtered and evaporated to dryness to give 6.2 g of crude material 14. The crude material was dissolved in MeOH (18 g), 35 mL of iPrOH were added and 20 g of solvent were removed under vacuum (41° C., 110 mbar). 15 mL of iPrOH were added to the suspension. After 1 h 5 g of solvent were removed under vacuum (41° C., 110 mbar), then 15 mL of iPrOH were added and the mixture was left 1 h at rt and 2 h at 4° C. 11 g of solvent were removed and finally 15 mL of iPrOH were added and the suspension was kept 18 h at 4° C. The crystals were filtered off, washed with 20 mL of iPrOH and dried under high vacuum to give 5.0 g of compound 14 as the mesylate salt with 1 equivalent of iPrOH.

¹H-NMR (CDCl₃, 400 MHz) δ: 12.8 (s, 1 H), 11.58 (brs, 1 H), 8.36 (brs, 1 H), 7.96 (s+brs, 2 H), 7.66 (s, 1 H), 7.55 (d+brs, J=8.4 Hz, 3 H), 7.34 (d, J=2 Hz, 1 H), 7.13 (s, 1 H), 4.36 (brs, 1 H), 3.93 (s, 3 H), 3.90 (s, 2 H), 3.88 (s, 3 H), 2.99 (s, 3 H), 2.76 (s, 3 H), 2.34 (s, 3 H, MeSO₃H). Rₜ (02)=1.33 minutes.

### Example 20

This example illustrates the preparation of 5-Chloro-3-[4-(2,4-diamino-pyrimidin-5-yl-methyl)-6,7-dimethoxy-benzofuran-2-carbonyl]-1 H-indole-2-carboxylic acid dimethyl-amide, mesylate salt 14 (one pot reaction of steps A6 and B6).

A slightly turbid solution of tBuOK (8.62 g, 97% content, 74.54 mmol, 1.05 eq.) in THF (170 mL, freshly distilled over sodium) was added dropwise over a period of 25 minutes to a vigorously stirred pale yellow solution of 6 (38.2 g, 95% content, 81.64 mmol, 1.15 eq.) in THF (330 mL, freshly distilled over sodium) 20° C. During the addition of the tBuOK solution an exothermic effect was observed (inside temperature rose slowly from 20° C. to 24° C.) and an intensive yellow suspension was formed. The mixture was stirred 90 minutes at 20° C. Compound 10 (37.2 g, 95% content, 70.99 mmol, 1 eq.) was added in one portion as a powder. The suspension was stirred for 90 minutes at 25° C. and a colour change from yellow to orange was observed. Then solid tBuOK (0.493 g, 97% content, 4.26 mmol, 0.06 eq.) was added as a powder. The mixture was stirred for further 90 minutes at 30° C. Sodium hydroxide solution (110 mL, 440 mmol, 4 M, 6.2 eq.) was added and the colour of the solution changed to dark yellow. The mixture was stirred for 135 minutes at 58° C. (inside temperature). The mixture was cooled to RT and the pH was lowered to approximately 6.5 by adding aqueous HCl solution (75 mL, 300 mmol, 4 M). NaHCO₃ saturated solution (700 mL) was added carefully, a minor formation of CO₂ was observed. The mixture was extracted with ethyl acetate/isopropanol 85/15 (2×1600 mL). The organic layers were washed with water/brine 90/10 (2×200 mL) and brine (1×200 mL), filtered through a plug of Celite, combined and evaporated to dryness. The resulting yellow foam was kept at high vacuum and RT for 16 hours.

The crude material (45 g, corresponds to 70.2 mmol) was dissolved in methanol (140 mL) to give a dark yellow solution. After addition of methanesulfonic acid (5.28 ml, 81.4 mmol, 1.146 eq.) at RT, isopropanol (224 mL) was added and the clear solution seeded. The mixture was stirred for lh at RT to form pale yellow crystals. Then a part of the solvents (about 150 mL) were slowly distilled off at 40° C. and 180 mbar. The precipitation became more intensive and slowly transformed into yellow crystals. The Suspension was stirred for 2 hours at 45° C. and then the slurry was slowly cooled to RT within 90 minutes. After stirring the suspension for 6 hours at RT the crystals were filtered and washed with isopropanol/methanol 95/5 (200 mL) to give 42.5 g yellow crystals of compound 14 as the mesylate salt with 1 equivalent of iPrOH.

¹H-NMR (CDCl₃, 400 MHz) δ: 12.8 (s, 1 H), 11.58 (brs, 1 H), 8.36 (brs, 1 H), 7.96 (s+brs, 2 H), 7.66 (s, 1 H), 7.55 (d+brs, J=8.4 Hz, 3 H), 7.34 (d, J=2 Hz, 1 H), 7.13 (s, 1 H), 4.36 (brs, 1 H), 3.93 (s, 3 H), 3.90 (s, 2 H), 3.88 (s, 3 H), 2.99 (s, 3 H), 2.76 (s, 3 H), 2.34 (s, 3 H, MeSO₃H). Rₜ (02)=1.33 minutes.

### Example 21

This example illustrates the preparation of 3-[4-(2,4-Bis-isobutyrylamino-pyrimidin-5-ylmethyl)-6,7-dimethoxy-benzofuran-2-carbonyl]-5-chloro-2-dimethylcarbamoyl-indole-1-carboxylic acid tert-butyl ester 15 (R=CH(CH₃)₂) (step A7).

A filtered solution of tBuOK (1.14 g, 0.95 eq.) in THF (10 mL) was added to a solution of 6 (5 g, 11.26 mmol, R=CH(CH₃)₂) in THF (40 mL). The mixture was stirred 30 minutes at RT before adding slowly 12 (4.75 g, 10.725 mmol). After 1 h, more tBuOK (184 mg, 0.15 eq.) was added. One hour later, the reaction was finished. The mixture was poured into a solution of HCl 0.05 N, extracted three times with AcOEt. The organic layers were washed with water and brine, then dried over MgSO₄, filtered and evaporated to dryness. Compound 15 (R=CH(CH₃)₂) was used for the next reaction without any further purification. Rₜ (01)=7.77 minutes.

### Example 22

This example illustrates the preparation of 3-{4-[2,4-Bis-(2,2-dimethyl-propionylamino)-pyrimidin-5-ylmethyl]-6,7-dimethoxy-benzofuran-2-carbonyl}-5-chloro-2-dimethylcarbamoyl-indole-1-carboxylic acid tert-butyl ester 15 (R=C(CH₃)₃) (step A7).

A filtered solution of tBuOK (452 mg, 0.95 eq.) in THF (3 mL) was added to a solution of 6 (2 g, 4.24 mmol, R=C(CH₃)₃) in THF (20 mL). The mixture is stirred 5 minutes at RT before adding dropwise a solution of 12 (1.88 g, 4.24 mmol) in THF (10 mL). After 1 h, more tBuOK (119 mg, 0.25 eq.) was added. After one hour, the mixture was poured into a solution of HCl 0.1 N, extracted three times with AcOEt. The organic layers were washed with water and brine, then dried over MgSO₄, filtered and evaporated to give crude 15 (R=C(CH₃)₃).

¹H-NMR (CDCl₃, 400 MHz) δ: 8.33 (s, 1 H), 8.20 (s, 1 H), 8.07 (s, 1 H), 8.01 (d, J=9.2 Hz, 1 H), 7.85 (brs, 2 H), 7.50 (d, J=2 Hz, 1 H), 7.19 (d, J=9.8 Hz, 1 H), 6.64 (s, 1 H), 3.92 (s, 3 H), 3.90 (s, 2 H), 3.75 (s, 3 H), 2.78 (s, 3 H), 2.71 (s, 3 H), 1.47 (s, 9 H), 1.12 (s, 9 H), 0.99 (s, 9 H). Rₜ (01)=8.15 minutes.

### Example 23

This example illustrates the preparation of 5-Chloro-3-[4-(2,4-diamino-pyrimidin-5-yl-methyl)-6,7-dimethoxy-benzofuran-2-carbonyl]-1H-indole-2-carboxylic acid dimethyl-amide, mesylate salt 14 (step B7).

The crude material 15 (R=CH(CH₃)₂) was dissolved in THF/H₂O (30 mL, 3/1), NaOH (2.15 g, 53.63 mmol) was added and the mixture was heated during 4 h at 50° C. The mixture was then poured into brine and extracted 3 times with AcOEt. The organic layers were washed with brine, dried over MgSO₄, filtered and evaporated to dryness to give 6.2 g of crude material 14. The crude material was dissolved in MeOH (18 g), 35 mL of iPrOH were added and 20 g of solvent were removed under vacuum (41° C., 110 mbar). 15 mL of iPrOH were added to the suspension. After 1 h 5 g of solvent were removed under vacuum (41° C., 110 mbar), then 15 mL of iPrOH were added and the mixture was left 1 h at rt and 2 h at 4° C. 11 g of solvent were removed and finally 15 mL of iPrOH were added and the suspension was kept 18 h at 4° C. The crystals were filtered off, washed with 20 mL of iPrOH and dried under high vacuum to give 5.3 g of compound 14 as the mesylate salt with 1 equivalent of iPrOH having the same NMR given in Example 19.

### Example 24

This example illustrates the preparation of 3-{4-[2,4-Bis-(2,2-dimethyl-propionyl-amino)-pyrimidin-5-ylmethyl]-6,7-dimethoxy-benzofuran-2-ylmethyl}-5-chloro-1-(toluene-4-sulfonyl)-1H-indole-2-carboxylic acid dimethylamide 16 (R=C(CH₃)₃) (step A8).

Compound 13 (0.065 g, 0.075 mmol, R=C(CH₃)₃) was dissolved in DCE (1 mL) and BF₃.OEt₂ (28 μL, 3 eq.) was added. After addition of triethyl-silane (36 μL, 3 eq.) the mixture was stirred at 85° C. for 1 h. After cooling to RT the reaction mixture was diluted with DCM (15 mL), washed with water (3 times 15 mL) and brine, dried over MgSO₄ and the solvent was distilled off. The crude mixture of compound 16 (R=C(CH₃)₃) was used in the next experiment without further purification.

### Example 25

This example illustrates the preparation of 3-{4-[2,4-Bis-(2,2-dimethyl-propionyl-amino)-pyrimidin-5-ylmethyl]-6,7-dimethoxy-benzofuran-2-ylmethyl}-5-chloro-2-dimethylcarbamoyl-indole-1-carboxylic acid tert-butyl ester 17 (R=C(CH₃)₃) (step A9).

Compound 15 (0.12 g, 0.147 mmol, R=C(CH₃)₃) was dissolved in DCE (2 mL) and BF₃.OEt₂ (55 gL, 3 eq.) was added. After addition of triethyl-silane (70 μL, 3 eq.) the mixture was stirred at 85° C. for 1 h. After cooling to RT the reaction mixture was diluted with DCM (15 mL), washed with water (3 times 15 mL) and brine, dried over MgSO₄ and evaporated to dryness. The crude mixture of compound 17 (R=C(CH₃)₃) was used in the next experiment without further purification.

### Example 26

This example illustrates the preparation of 5-Chloro-3-[4-(2,4-diamino-pyrimidin-5-yl-methyl)-6,7-dimethoxy-benzofuran-2-ylmethyl]-1 H-indole-2-carboxylic acid dimethyl-amide I (step A10).

The mesylate salt 14 (4.88 g, 6.932 mmol) was dissolved in water (50 mL). AcOEt (50 mL) was added, and then the mixture was quenched with NaHCO₃ 10% (50 mL) and stirred vigorously. The organic layers were separated and the aqueous layers were extracted with AcOEt (50 mL). The combined organic layers were washed with water (100 mL), brine (50 mL) and dried over MgSO₄, filtered and evaporated to dryness. iPrOH (30 mL) was added to the yellowish compound, then NaBH₄ (352 mg, 9.317 mmol) was added and the mixture heated at 50° C. during 3 h. Water (50 mL) was added and the mixture was extracted 3 times with AcOEt (50 mL each). Organic layers were washed with NaOH 0.2 N (50 mL), then with brine (50 mL), dried over MgSO₄, filtered and evaporated to dryness. It was then recrystallized from iPrOH to give 2.75 g of compound 1.

¹H-NMR (CDCl₃, 400 MHz) δ: 11.64 (s, 1 H), 7.69 (s, 1 H), 7.37 (d, J=8.8 Hz, 1 H), 7.33 (s, 1 H), 7.15 (dd, J₁=8.4 Hz, J₂=2 Hz, 1 H), 6.80 (s, 2 H), 6.47 (s, 2 H), 6.09 (brs, 1 H), 5.67 (brs, 1 H), 4.20 (s, 2 H), 3.86 (s, 3 H), 3.74 (s, 3 H), 3.66 (s, 2 H), 2.94 (s, 6 H).

Rₜ (01)=4.51 minutes. Rₜ (02)=1.45 minutes.

### Example 27

This example illustrates the preparation of 5-Chloro-3-[4-(2,4-diamino-pyrimidin-5-yl-methyl)-6,7-dimethoxy-benzofuran-2-ylmethyl]-1 H-indole-2-carboxylic acid dimethyl-amide I (step A10).

The mesylate salt of compound 14 (+1 iPrOH) (1.7 g, 2.41 mmol) was suspended in iPrOH (6 mL) and dimethoxyethane (4 mL). This slurry was cooled with an Ice-bath (0° C., 30 minutes), then NaBH₄ (364 mg, 9.64 mmol, 4 eq.) was slowly added (strong H₂ evolution). After 5 minutes a clear yellow solution appeared. After 2 hr 14 was fully reduced to the alcohol 14A. The solution was warmed to 35° C. and kept at this temperature for 5 hrs. The reaction mixture was cooled with an ice-bath and 1 N HCl (15 mL) was slowly added. The solution was stirred at RT for 1 h. Half of the organic solvents were removed and ethylacetate (20 mL) was added. After addition of 5% NaOH (6 mL) and saturated NaCl (10 mL) the layers were separated and the aqueous layer extracted with additional ethylacetate (20 mL). The organic layers were washed with saturated NaCl (20 mL). The organic layers were filtered through Celite and evaporated to dryness (1.41 g). The yellow foam was dissolved in methanol (2 mL), seeded and crystallized for 24 h. The crystals were filtered and washed with 10 mL iPrOH/MeOH (1:1). The crystals were dried at the HV/RT to yield 1.2 g of compound I having the same NMR given in Example 26.

### Example 28

This example illustrates the preparation of 5-Chloro-3-[4-(2,4-diamino-pyrimidin-5-yl-methyl)-6,7-dimethoxy-benzofuran-2-ylmethyl]-1 H-indole-2-carboxylic acid dimethyl-amide I (step A10).

The mesylate salt 14 (6% isopropanol) (20.0 g, 29.8 mmol) was suspended in tetrahydrofuran (THF) (200 mL) at RT. This slurry was cooled with an ethanol bath equipped with a Cryocool (−25° C., 1 h), then Zn(BH₄)₂ (1,5 mol eq, 1.5 M solution in THF, 30 mL) was slowly added dropwise (some H₂ evolution) in 3 portions (3×10 mL every 15 minutes). After additional 15 minutes of stirring the slurry was warmed to 0° C. HCl solution was added continuously (1 eq, 4 M solution in dioxane, 7.45 mL, 124 μL/min) over a 1 h period. The solution was stirred for additional 15 minutes at 0° C. and then allowed to warm up to 20° C. during a period of 2 h. The clear yellow solution was cooled to 0° C. with an ice-bath and water (80 mL) was slowly added dropwise over a 25 minutes period. One hour later, HCl 37% in water (100 mL) was added dropwise at 0° C. over 10 minutes and the solution was stirred for 14 hours at RT. The reaction mixture was cooled to 0° C. with an ice-bath and 10 N NaOH (140 mL) was slowly added. The solution showed a pH value of approximately 9. The pale yellow solution was 3 times extracted with a mixture of ethylacetate/isopropanol (85:15, 3×400 mL). The organic layers were continued washed with water/10 N NaOH (80:20, 100 mL), water/sat. NaCl (10:40, 50 mL) and 2 times water/sat. NaCl (5:45, 50 mL). The organic layers were dried with sodium sulfate and evaporated to dryness.

The yellow solid was stirred in methanol (30 mL) at 45° C. for 3 hours where the foam was converted into a crystalline precipitate. Then the slurry was stirred for 24 h at RT. The crystals were filtered and washed with 20 mL methanol and dried at HV/RT for 14 h to give 13.56 g of compound I having the same NMR given in Example 26.

### Example 29

This example illustrates the preparation of 5-Chloro-3-{[4-(2,4-diamino-pyrimidin-5-ylmethyl)-6,7-dimethoxy-benzofuran-2-yl]-hydroxy-methyl}-1H-indole-2-carboxylic acid dimethylamide 14A (step B10).

The mesylate salt of the compound 14 (100 mg, 0.155 mmol) was dissolved in a mixture of iPrOH/MeOH (2/0.5 mL). The reaction mixture was cooled to −20° C., before addition of sodium borohydride (17.6 mg, 0.466 mmol). The mixture was stirred during 1 h at −20° C., then NaOH 0.1 N (5 mL) was added. The mixture was extracted 3 times with AcOEt (15 mL each). The organic layers were washed with brine (50 mL), dried over MgSO₄, filtered and evaporated to dryness. The crude mixture containing compound 14A was directly used in the next step. Rₜ (02)=1.32 minutes.

### Example 30

This example illustrates the preparation of 5-Chloro-3-[4-(2,4-diamino-pyrimidin-5-yl-methyl)-6,7-dimethoxy-benzofuran-2-ylmethyl]-1 H-indole-2-carboxylic acid dimethyl-amide I (step C10).

The secondary alcohol 14A (50 mg, 90.9 μmol) was dissolved in THF (2 mL). The reaction mixture was cooled to -20° C., before addition of sodium borohydride (10.3 mg, 0.273 mmol). After 5 minutes stirring at −20° C., BF₃.OEt₂ (34 μL, 50%) was added slowly. After each drop of BF₃.OEt₂, the color of the mixture was turning to violet, then the violet color disappeared again. After complete addition of BF₃.OEt₂, the violet color was persistent during 3 minutes before returning to a pale yellow solution. The reaction was complete after 5 minutes and then NaOH 0.1 N (10 mL) was added. The mixture was extracted 2 times with EtOAc (15 mL each). The organic layers were washed with brine (50 mL), dried over MgSO₄, filtered and evaporated to dryness to give the final compound I having the same LCMS signals given in Example 26.

### Example 31

This example illustrates the preparation of 5-Chloro-3-[4-(2,4-diamino-pyrimidin-5-yl-methyl)-6,7-dimethoxy-benzofuran-2-ylmethyl]-1H-indole-2-carboxylic acid dimethyl-amide I (step A11).

The crude material of Example 20 (compound 16, R=C(CH₃)₃) was dissolved in methanol (2 mL) and after addition of 4 N NaOH (10 eq.) the solution was stirred for 3 h at 50° C. After cooling to RT the mixture was extracted 2 times with EtOAc (20 mL each). The organic layers were washed with brine (50 mL), dried over MgSO₄, filtered and evaporated to dryness to give the final compound I having the same LCMS signals given in Example 26.

### Example 32

This example illustrates the preparation of 5-Chloro-3-[4-(2,4-diamino-pyrimidin-5-yl-methyl)-6,7-dimethoxy-benzofuran-2-ylmethyl]-1H-indole-2-carboxylic acid dimethyl-amide I (step A12).

The crude material of Example 21 (compound 17, R=C(CH₃)₃) was dissolved in methanol (4 mL) and after addition of 4 N NaOH (10 eq.) the solution was stirred for 3 h at 50° C. After cooling to RT the mixture was extracted 2 times with EtOAc (30 mL each). The organic layers were washed with brine (50 mL), dried over MgSO₄, filtered and evaporated to dryness to give the final compound I having the same LCMS signals given in Example 26.

1. A process for manufacturing a compound of formula I, 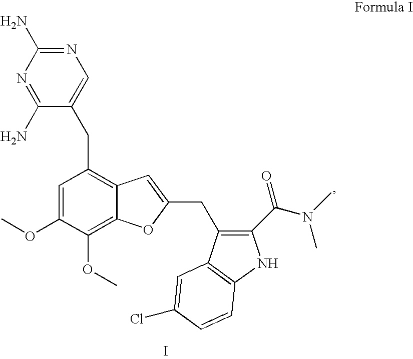
comprising:
starting with a compound of formula 6, 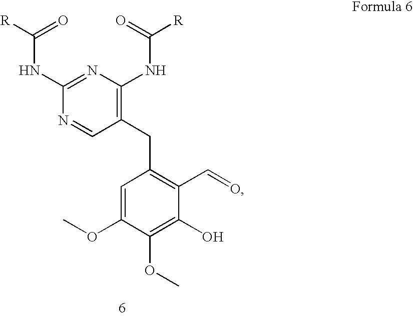
wherein R represents —C(CH₃)₃ or —CH(CH₃)₂;
reacting the compound of formula 6 with either a compound of formula 10 or 12, 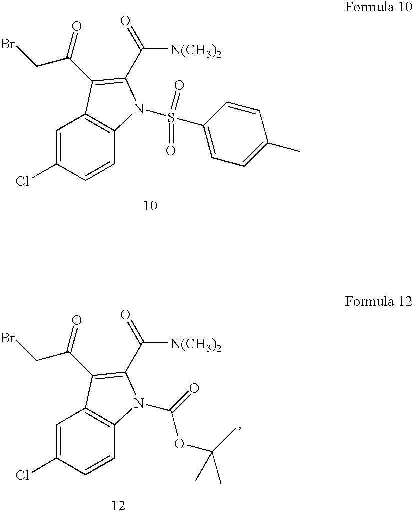
to obtain a compound of formula 13 or 15, respectively; 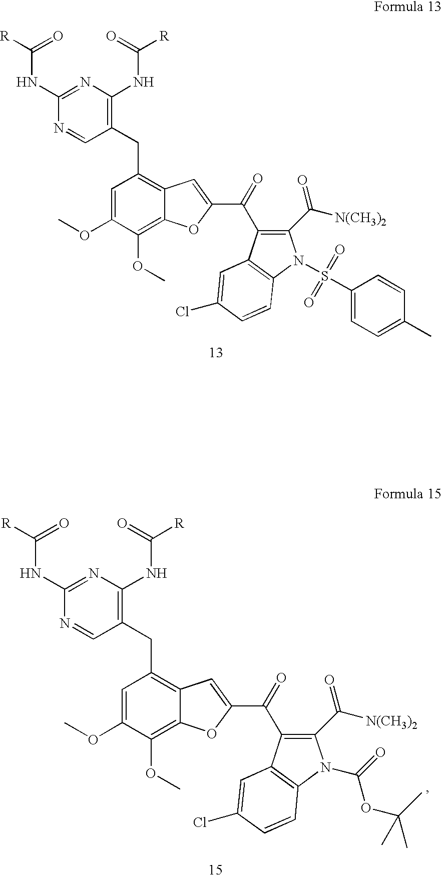
wherein R has the meaning given in formula 6 above;
deprotecting the compound of formula 13 or 15 to obtain a compound of formula 14, 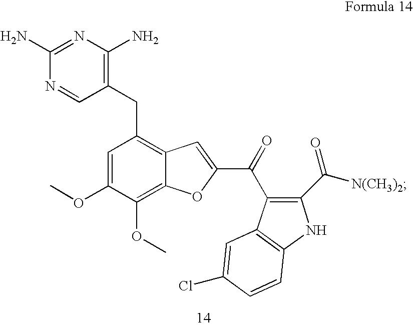
and transforming the keto carbonyl group of the compound of formula 14 by a one- or two-step reduction to obtain the compound of formula I.

2. A method for preparing a compound of formula I, 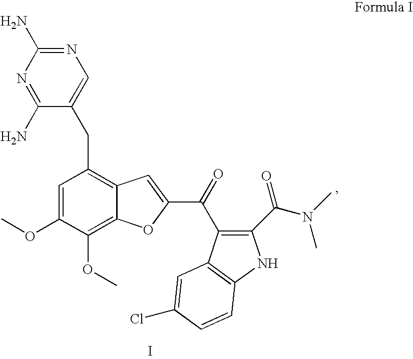
comprising:
reducing the compound of formula 14, 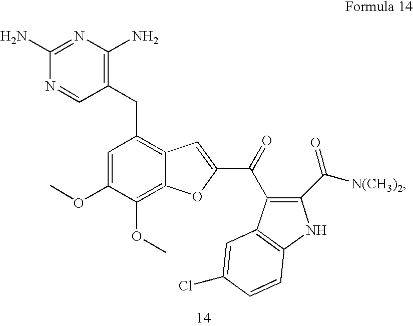
with either a borohydride in a temperature range of −20 C. up to 70° C., depending on the borohydride applied,
or in a two-step reduction, reducing the compound of formula 14 via a compound of formula 14A, 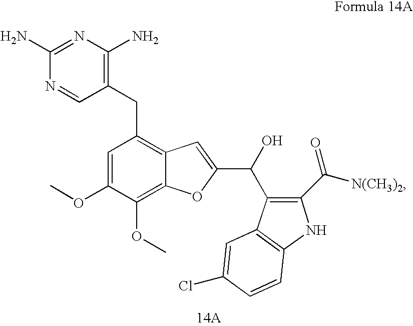
using, in a first step, sodium borohydride at −20° C. or a ruthenium catalyst at room temperature and, in a second step, sodium borohydride at about 0° C. with boron trifluoride or trifluoroacetic acid as a catalyst, to obtain the compound of formula I.

3. A compound selected from the group consisting of:
N-[4-(2,2-Dimethyl-propionylamino)-5-(3,4,5-trimethoxy-benzyl)-pyrimidin-2-yl]-2,2-dimethyl-propionamide;
N-[4-Isobutyrylamino-5-(3,4,5-trimethoxy-benzyl)-pyrimidin-2-yl]-isobutyramide;
N-[4-(2,2-Dimethyl-propionylamino)-5-(2-formyl-3,4,5-trimethoxy-benzyl)-pyrimidin-2-yl]-2,2-dimethyl-propionamid;
N-[5-(2-Formyl-3,4,5-trimethoxy-benzyl)-4-isobutyrylamino-pyrimidin-2-yl]-isobutyramide;
N-[4-(2,2-Dimethyl-propionylamino)-5-(2-iodo-3,4,5-trimethoxy-benzyl)-pyrimidin-2-yl]-2,2-dimethyl-propionamide;
N-[5-(2-Iodo-3,4,5-trimethoxy-benzyl)-4-isobutyrylamino-pyrimidin-2-yl]-isobutyramide;
N-[4-(2,2-Dimethyl-propionylamino)-5-(2-formyl-3-hydroxy-4,5-dimethoxy-benzyl)-pyrimidin-2-yl]-2,2-dimethyl-propionamide;
N-[5-(2-Formyl-3-hydroxy-4,5-dimethoxy-benzyl )-4-isobutyrylamino-pyrimidin-2-yl]-isobutyramide;
3-Acetyl-5-chloro-1H-indole-2-carboxylic acid dimethylamide;
3-Acetyl-5-chloro-1-(toluene-4-sulfonyl)-1H-indole-2-carboxylicacid dimethylamide;
3-(2-Bromo-acetyl)-5-chloro-1-(toluene-4-sulfonyl)-1H-indole-2-carboxylicacid-dimethylamide;
3-(2-Bromo-acetyl)-5-chloro-1H-indole-2-carboxylicacid-dimethylamide;
3-(2-Bromo-acetyl)-5-chloro-2-dimethylcarbamoyl-indole-1-carboxylic acid tert-butyl ester;
3-[4-(2,4-Bis-isobutyrylamino-pyrimidin-5-ylmethyl)-6,7-dimethoxy-benzofuran-2-carbonyl]-5-chloro-1-(toluene-4-sulfonyl)-1H-indole-2-carboxylicacid-dimethylamide;
3-{4-[2,4-Bis-(2,2-dimethyl-propionyl-amino)-pyrimidin-5-ylmethyl]-6,7-dimethoxy-benzofuran-2-ylmethyl}-5-chloro-1-(toluene-4-sulfonyl)-1H-indole-2-carboxylic acid dimethylamide;
5-Chloro-3-[4-(2,4-diamino-pyrimidin-5-yl-methyl)-6,7-dimethoxy-benzofuran-2-carbonyl]-1H-indole-2-carboxylic acid dimethyl-amide, mesylate salt;
3-[4-(2,4-Bis-isobutyrylamino-pyrimidin-5-ylmethyl)-6,7-dimethoxy-benzofuran-2-carbonyl]-5-chloro-2-dimethylcarbamoyl-indole-1-carboxylic acid tert-butyl ester;
3-{4-[2,4-Bis-(2,2-dimethyl-propionylamino)-pyrimidin-5-ylmethyl]-6,7-dimethoxy-benzofuran-2-carbonyl}-5-chloro-2-dimethylcarbamoyl-indole-1-carboxylic acid tert-butyl ester;
3-{4-[2,4-Bis-(2,2-dimethyl-propionyl-amino)-pyrimidin-5-ylmethyl]-6,7-dimethoxy-benzofuran-2-ylmethyl}-5-chloro-1-(toluene-4-sulfonyl)-1H-indole-2-carboxylic acid dimethylamide;
3-[4-(2,4-Bis-isobutyrylamino-pyrimidin-5-ylmethyl)-6,7-dimethoxy-benzofuran-2-ylmethyl]-5-chloro-1-(toluene-4-sulfonyl)-1H-indole-2-carboxylic acid dimethyl-amide;
3-{4-[2,4-Bis-(2,2-dimethyl-propionyl-amino)-pyrimidin-5-ylmethyl]-6,7-dimethoxy-benzofuran-2-ylmethyl}-5-chloro-2-dimethylcarbamoyl-indole-1-carboxylic acid tert-butyl ester; and
3-[4-(2,4-Bis-isobutyrylamino-pyrimidin-5-ylmethyl)-6,7-dimethoxy-benzofuran-2-ylmethyl]-5-chloro-2-dimethylcarbamoyl-indole-1-carboxylic acid tert-butyl ester.
